(19) United States
(12) Patent Application Publication
(10) Pub. No.: US 2014/0204334 A1
(43) Pub. Date: Jul. 24, 2014

(54) Bio-sensors in eyeglasses

(71) Applicant: William Anthony Stoll, Ellicottville, NY (US)

(72) Inventor: William Anthony Stoll, Ellicottville, NY (US)

(21) Appl. No.: 13/694,914

(22) Filed: Jan. 18, 2013

(51) Int. Cl. G02C 11/00; A61B 5/024; A61B 5/145

(52) U.S. Cl. CPC G02C 11/10; A61B 5/14532; A61B 5/145; A61B 5/024
USPC 351/158; 600/365; 600/309; 600/500

(57) ABSTRACT

A wearable medical device (10) comprising: spectacles or eyeglasses (12) including at least one temple piece (14) and nose-engagers (16); at least one bio-sensor (18) for measuring at least one physical parameter of a person wearing said spectacles (12), said bio-sensor (18) being associated with one of said temple piece (14) or said nose-engagers (16); information storage (20) incorporated with said spectacles (12) for storing information received from said bio-sensor (18); a transmitter (22) for transmitting said stored information; and a power supply (24) for driving said bio-sensor, said storage and said transmitter.

### CROSS-REFERENCE TO RELATED APPLICATIONS

This application claims priority from Provisional Patent Application Ser. No. 61/632,506, filed Jan. 25, 2012.

### GOVERNMENT CONTRACT

This invention was not made under any government contract and the United States Government has no rights under this invention.

1. Technical Field

This invention relates to patient monitoring via wearable biosensors, particularly, eyeglasses.

2. Background Art

An increasing number of wearable bio-sensors in the form of shirts, rings, wrist mounts or belt-type arrangements have recently been proposed. International Patent Application WO 2006/023341 A2 discloses eyeglasses with an included audio unit. U.S. Pat. No. 6,542,081 B2 discloses eyeglasses including apparatus for monitoring eye movement. U.S. Pat. No. 7,286,287 B1 discloses eyeglasses including a visual aid in the form of telescopic spectacles with an automated focusing device. And, U.S. Pat. No. 6,774,795 B2 discloses a cane or walking stick or other personal device including electronic functions. While all of these devices work to some degree, they tend to be bulky and/or difficult and/or embarrassing to use or are designed for a specific purpose. For example, the above-mentioned U.S. Pat. No. 7,286,287 B1 provides increased visual acuity during vascular surgery.

It would be an advance in the art to provide wearable eyeglasses to monitor critical patient criteria. It would be an advance also to provide a wearable monitor whereby the wearer could check blood alcohol levels in a relatively private manner.

### DISCLOSURE OF INVENTION

It is an object of the invention to enhance wearable bio-sensors.

Yet another object of the invention is the improvement of wearable bio-sensors.

These object are accomplished, in one aspect of the invention, by a wearable medical device comprising: spectacles including at least one temple piece and nose-engagers; at least one bio-sensor for measuring at least one physical parameter of a person wearing the spectacles. The bio-sensor associated with one of the temple piece or the nose-engagers can include information storage incorporated with the spectacles for storing information received from the bio-sensor; a transmitter for transmitting the stored information; and a power supply for driving the bio-sensor, the storage and the transmitter. Additionally, a concealed read-out display can be provided whereby the wearer can make a decision based upon the information in the display.

### BRIEF DESCRIPTION OF THE DRAWINGS

FIG. 1 is a perspective view of an embodiment of the invention;

FIG. 2 is an elevation view of an embodiment of the invention in place on a wearer; and

FIG. 3 is an elevation view of an alternate embodiment of the invention.

### BEST MODE FOR CARRYING OUT THE INVENTION

For purposes of this application it is to be understood that when an element or layer is referred to as being “on,” “connected to” or “coupled to” another element or layer, it can be directly on, connected to or coupled to the other element or layer or intervening elements or layers may be present. In contrast, when an element is referred to as being “directly on,” “directly connected to” or “directly coupled to” another element or layer, there are no intervening elements or layers present. Like numbers refer to like elements throughout. The term “and/or” includes any and all combinations of one or more of the associated listed items.

Although the terms “first,” “second,” “third” etc. may be used to describe various elements, components, regions, layers and/or sections, these elements, components, regions, layers and/or sections are not to be limited by theses terms as they are used only to distinguish one element, component, region, layer or section from another element, component, region, layer or section. Thus, a first element, component, region, layer or section could be termed a second element, component, region, layer or section without departing from the scope and teachings of the present invention.

Spatially relative terms, such as “beneath,” below,” upper,” “lower,” “above” and the like may be used herein for ease of description to describe one element or feature's relationship to another element(s) or feature(s) as illustrated in the drawings. These spatially relative terms are intended to encompass different orientations of the device in use or operation in addition to the orientation shown in the drawings. For example, if the device in the drawings is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of the invention. For example, as used herein, the singular forms “a,” “an” and “the” are intended to include the plural forms as well, unless the context clearly indicates otherwise. It will be further understood that the terms, “comprises” and/or “comprising,” when used in this specification, specify the presence of stated features, integers, steps operations, elements, and/or components, but do not preclude the presence or addition of one or more other features, integers, steps, operations, elements, components, and/or groups thereof.

For a better understanding of the present invention, together with other and further objects, advantages and capabilities thereof, reference is made to the following disclosure and appended claims taken in conjunction with the above-described drawings.

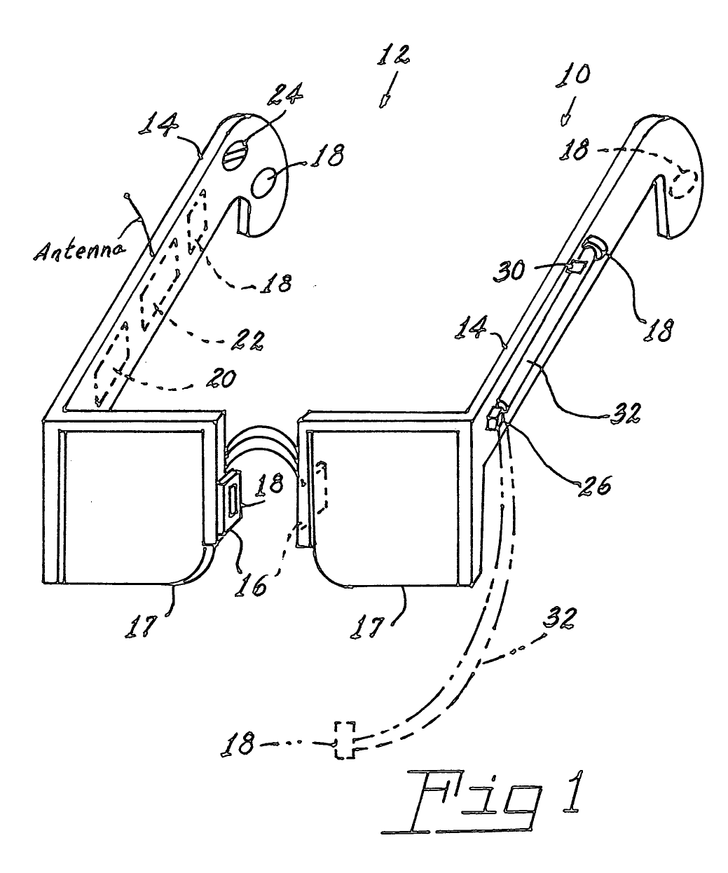

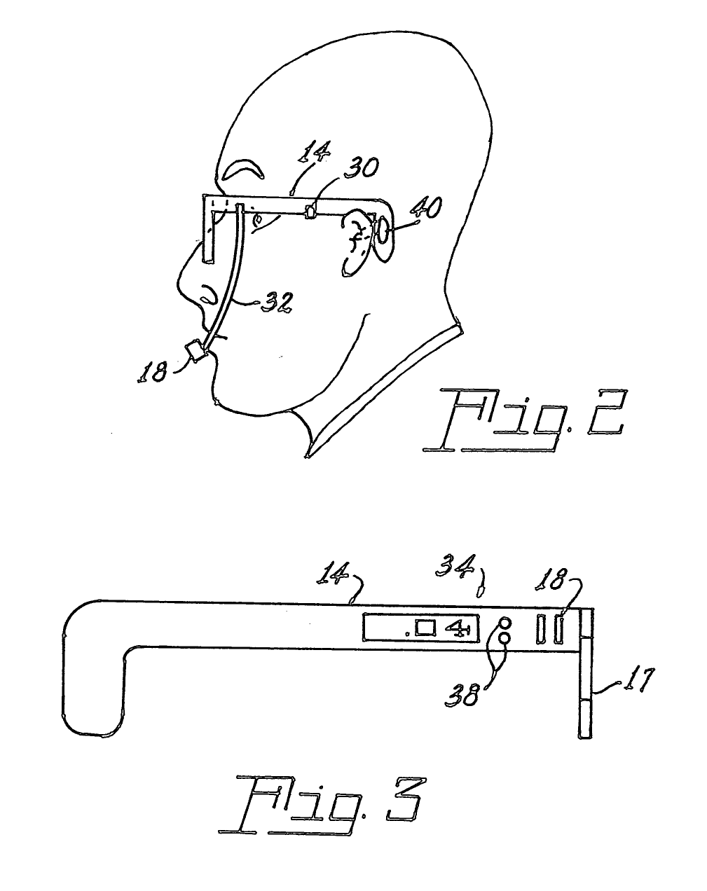

Referring now to the drawings with greater particularity there is shown in FIG. 1 a wearable medical device 10 in the form of spectacles 12 including at least one temple piece 14 and nose-engagers 16. Lenses 17 can be provided and they may, of course, be of the vision-correctable variety if necessary. At least one bio-sensor 18 for measuring at least one physical parameter of a person wearing the spectacles 12 is provided. The bio-sensors 18 can be associated with one of said temple pieces 14, either on the outer portion, i.e., that side facing away from the wearer, as shown in FIGS. 1 and 2, or the inner portion as shown in FIG. 3. Also, the bio-sensor or sensors 18 can be associated with the nose-engagers 16 as shown in FIG. 1. Information storage 20 can be incorporated with the spectacles 12 for storing information received from the bio-sensor 18, for example, in an area of the temple 14. A transmitter 22 also can be incorporated within the temple piece 14 for transmitting the stored information to a remote location and an antenna 28 can be provided to aid in the transmission. A power supply 24, which can be in the form of one or more batteries is provided for driving the bio-sensors 18, the storage 20 and the transmitter 24. While the power supply 24 can be remote from the spectacles 12, preferably it is located within the spectacle frame, for example, in the temple piece 14.

In an embodiment the at least one bio-sensor 18 can be associated with the temple piece in the area of the temple piece 14 that is normally behind the ear of the wearer.

In yet another embodiment the at least one bio-sensor 18 can be associated with the temple piece 14 by attachment thereto via a hinge 26 and an elongated tube 32. In this instance the bio-sensor 18 can be responsive to the breath of the wearer and, in use, can be removed from its locking mechanism 30 and pivoted about the hinge 26 to a position adjacent the mouth of the wearer, as illustrated in phantom lines in FIG. 1 and solid lines in FIG. 2.

In yet another embodiment an audio receiver in the form of a pivotable ear bud 40 can be attached to one of the temple pieces 14, as shown in FIG. 2

A number of parameters of the wearer are measurable by the device of the invention. By way of example, the bio-sensor or bio-sensors 18 can measure the blood glucose levels of the wearer; the blood alcohol level of the wearer, or the pulse rate of the wearer.

In the case of measuring the blood alcohol level of the wearer an indicator can be provided to indicate whether or not the wearer should be driving a motor vehicle.

Further, to protect the privacy of the wearer in the case of testing for blood alcohol levels, it would be advantageous for the wearer to be able to test inconspicuously. To accomplish this test the embodiment of the invention shown in FIG. 3 would be advantageous. Therein, the temple piece 14 is provided with a bio-sensor 18 on the interior side of the temple piece 14 adjacent to the lens 17. An indicator 34 can be provided next to the bio-sensor 18. The indicator 34 can provide a digital readout via a meter 36 or a GO/NOGO indication via red and green LEDs 38. In the case of a blood alcohol detector the bio-sensor can employ the well-known reaction of the oxidation of ethanol to acetic acid and water. The electrical current produced by this reaction is measured by a microprocessor and displayed as desired. In this case the wearer can test for a blood alcohol content by removing the spectacles and blowing in the bio-sensor 18 in a manner similar to that employed by many people to clean their lenses

While there have been shown and described what are at present considered to be the preferred embodiments of the invention, it will be apparent to those skilled in the art that various changes and modifications can be made herein without departing from the scope of the invention as defined by the appended claims.

### GLOSSARY OF REFERENCE NUMERALS EMPLOYED IN THIS APPLICATION

10 Wearable device

12 Eyeglasses

14 Temple piece

16 Nose-engagers

17 lens

18 Bio-sensor

20 Information storage

22 Transmitter

24 Power supply

26 Hinge

30 Locking mechanism

32 Elongated tube

34 Indicator

36 Meter

38 LEDs

40 Audio receiver

What is claimed is:

1. A wearable medical device (10) comprising:
spectacles or eyeglasses (12) including at least one temple piece (14) and nose-engagers (16);
at least one bio-sensor (18) for measuring at least one physical parameter of a person wearing said spectacles (12), said bio-sensor (18) being associated with one of said temple piece (14) or said nose-engagers (16);
information storage (20) incorporated with said spectacles (12) for storing information received from said bio-sensor (18);
a transmitter (22) for transmitting said stored information; and
a power supply (24) for driving said bio-sensor, said storage and said transmitter.

2. The wearable medical device (10) of claim 1 wherein said at least one biosensor (18) is associated with said nose-engagers (16).

3. The wearable medical device (10) of claim 1 wherein said at least one bio-sensor (18) is associated with said temple piece.

4. The wearable medical device (10) of claim 3 wherein said at least one bio-sensor (18) associated with said temple piece (14) is attached to said temple piece (14) by a hinge (26).

5. The wearable medical device (10) of claim 4 wherein said bio-sensor (18) is responsive to the breath of the wearer.

6. The wearable medical device (10) of claim 1 wherein said bio-sensor (18) measures the blood glucose levels of the wearer.

7. The wearable medical device (10) of claim 1 wherein said bio-sensor (18) measures the blood alcohol level of the wearer.

8. The wearable medical device (10) of claim 1 wherein said bio-sensor (18) measures the pulse rate of the wearer.

9. The wearable medical device (10) of claim 7 wherein said bio-sensor (18) is positioned on the inside of temple piece (14).

10. The wearable medical device (10) of claim 9 wherein an indicator (34) is positioned adjacent said bio-sensor (18).
